(19) United States
(12) Patent Application Publication
(10) Pub. No.: US 2024/0057987 A1
(43) Pub. Date: Feb. 22, 2024

(54) IMPLANT-CINCHING DEVICES AND SYSTEMS

(71) Applicant: Edwards Lifesciences Innovation (Israel) Ltd., Caesarea (IL)

(72) Inventors: Tal Sheps, Givat Shmuel (IL); Yaron Keidar, Kiryat Ono (IL); Yuval Zipory, Modi'in (IL)

(21) Appl. No.: 18/497,951

(22) Filed: Oct. 30, 2023

Related U.S. Application Data
(63) Continuation of application No. 17/097,649, filed on Nov. 13, 2020, now Pat. No. 11,832,784.
Application No. 17/097,649 is a continuation of application No. 16/154,233, filed on Oct. 8, 2018, now Pat. No. 10,835,221.
(60) Provisional application No. 62/580,646, filed on Nov. 2, 2017.

(51) Int. Cl. A61B 17/00; A61B 17/12; A61F 2/24

(52) U.S. Cl. CPC A61B 17/00234; A61B 17/0057; A61B 17/12013; A61F 2/2445; A61F 2/2466; A61B 2017/00243; A61F 2210/0057; A61F 2220/0016; A61B 2017/0496

(57) ABSTRACT

An implant comprises an elongate member, and anchors distributed along the elongate member. A gripper comprises a locking element that, in a locked state thereof, inhibits sliding of the elongate member through the gripper by clamping the elongate member. A tool is configured to, while (i) the elongate member extends proximally from a proximal-most of the anchors, and through the gripper, and (ii) an unlocker is positioned within the gripper in a manner that obstructs the locking element from assuming the locked state: (A) advance transluminally toward the proximal-most anchor, and (B) tension the elongate member by pulling the elongate member proximally through the gripper while the unlocker obstructs the locking element from assuming the locked state. The tool is also configured to subsequently facilitate the locking element clamping the elongate member via withdrawal of the unlocker from the gripper. Other embodiments are also described.

### CROSS-REFERENCES TO RELATED APPLICATIONS

The present application is a Continuation of U.S. Ser. No. 17/097,649 to Sheps et al., filed Nov. 13, 2020, and entitled “Implant-cinching devices and systems;” which is a Continuation of U.S. Ser. No. 16/154,233 to Sheps et al., filed Oct. 8, 2018, and entitled “Implant-cinching devices and systems” (now U.S. Pat. No. 10,835,221); which claims priority from U.S. Provisional application 62/580,646 to Sheps et al., filed Nov. 2, 2017, and entitled “Implant-cinching devices and systems.”

Each of the above applications is incorporated herein by reference.

### FIELD OF THE INVENTION

Some applications of the present invention relate in general to adjustment of an implant. For example, some applications of the present invention relate to contraction of a cardiovascular implant.

### BACKGROUND

Ischemic heart disease can cause valve regurgitation, such as mitral regurgitation by the combination of ischemic dysfunction of the papillary muscles, and the dilatation of the left ventricle that is present in ischemic heart disease, with the displacement of the papillary muscles and the dilatation of the mitral valve annulus.

Dilation of the annulus of the mitral valve (or another valve) can prevent the valve leaflets from fully coapting when the valve is closed. Mitral regurgitation of blood from the left ventricle into the left atrium results in increased total stroke volume and decreased cardiac output, and ultimate weakening of the left ventricle secondary to a volume overload and a pressure overload of the left atrium.

Dilation of other regions of the heart, vascular system, and/or other valve annuluses can also result in similar problems, including regurgitation at other valves.

Annuloplasty, such as by implantation of an annuloplasty ring or other annuloplasty device, can be used to improve leaflet coaptation by adjusting the shape of a native valve annulus, e.g., the mitral annulus, tricuspid annulus, etc.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features described can be combined in a variety of ways. The description herein relates to systems, assemblies, methods, devices, apparatuses, combinations, etc. that may be utilized for cinching, tightening, tensioning a medical implant. Various features and steps as described elsewhere in this disclosure may be included in the examples summarized here.

As one example, an adjustment system can include an elongate member. The elongate member can enter into a pouch (e.g., an elastic pouch) at a first part of the pouch, extend through the pouch, and can be connected to a second part of the pouch. The system and its components can be configured such that stretching of the pouch (e.g., elastically stretching the pouch) causes a distance between the first and second parts of the pouch to increase, thereby drawing more of the elongate member into the pouch. The system can include a gripper. The gripper can be disposed at the first part of the pouch, and can be configured such that it can inhibit the elongate member from re-exiting the pouch when the pouch is allowed to re-contract. Therefore, the net result of stretching and releasing the pouch can be reduction of a length of the elongate member that is disposed outside of the pouch.

The elongate member can be attached to or be part of an implant or implant body in a manner that changes a dimension of the implant or implant body when the length of the elongate member that is disposed outside of the pouch is reduced. For some applications, the implant or implant body includes a sleeve that is longitudinally contracted when the length of the elongate member that is disposed outside of the pouch is reduced. For some such applications, the implant or implant body can be an annuloplasty ring structure, other annuloplasty device structure, or other cinchable/tensionable structure, anchored to the native valve annulus (e.g., mitral valve annulus, tricuspid valve annulus, etc.), and longitudinal contraction of the sleeve contracts the native valve annulus. The implant or implant body can have an open or closed (e.g., a closed ring) configuration and can be configured for transvascular or transcatheter implantation and/or surgical implantation.

A system or apparatus for tightening, cinching, or tensioning can include one or more of a gripper, an elastic pouch, and an elongate member. The elastic pouch can have a first part and a second part. The first part can be coupled to the gripper. The elastic pouch can be stretchable into a stretched state in which the pouch defines a stretched distance between the first part and the second part. The elastic pouch can also have a contracted state toward which the pouch is elastically biased, and in which the pouch defines a contracted distance between the first part and the second part, the contracted distance being smaller than the stretched distance. In at least one state of the gripper, the elongate member can be slidable through the gripper and into the pouch.

The system or apparatus above and/or another similar system or apparatus can include one or more of an implant having an implant body, a gripper, coupled to the implant body, an elastic pouch, and an elongate member. The elastic pouch can be coupled to the gripper, have a first part and a second part, be reversibly stretchable into a stretched state in which the pouch defines a stretched distance between the first part and the second part, and/or have a contracted state toward which the pouch is elastically biased, and in which the pouch defines a contracted distance between the first part and the second part, the contracted distance being smaller than the stretched distance.

The elongate member can have a first end portion coupled to the implant body, a second end portion fastened to the second part of the pouch, and a third portion or a mid-portion extending (i) from the second end portion, through the pouch to the first part of the pouch, and (ii) out of the pouch to the first end portion. In at least one state of the gripper, the third portion or mid-portion can be slidable through the gripper and into the pouch.

In an application, the implant or implant body can include an annuloplasty ring structure, other annuloplasty device structure, or other cinchable/tensionable structure.

In an application, the gripper includes a plurality of teeth that provide a gripping surface configured to grip the elongate member.

In an application, the pouch is coupled to the gripper by the first part of the pouch being coupled to the gripper.

In an application, the first part of the pouch defines an opening into the pouch, and in the at least one state of the gripper, the third portion or mid-portion in slidable through the gripper and into the pouch via the opening.

In an application, in at least one state of the gripper, the gripper inhibits sliding of the third portion or mid-portion through the gripper and out of the pouch.

In an application, the at least one state of the gripper includes a unidirectional state in which the gripper facilitates sliding of the third portion or mid-portion through the gripper in a first direction that is into the pouch, and inhibits sliding of the third portion or mid-portion through the gripper in a second, opposite direction that is out of the pouch.

In an application, the gripper has an unlocked state in which the gripper facilitates sliding of the third portion or mid-portion through the gripper in the first direction and in the second direction.

In an application, the gripper includes one or more wheels that, in the unidirectional state, grip the third portion or mid-portion, each of the one or more wheels configured to rotate in only one rotational direction.

In an application, the at least one state of the gripper includes:
an unlocked state in which the gripper facilitates sliding of the third portion or mid-portion through the gripper in a first direction that is into the pouch, and in a second, opposite direction that is out of the pouch, and
a locked state in which the gripper inhibits sliding of the third portion or mid-portion through the gripper in the first direction and in the second direction.

In an application, the apparatus further includes an unlocker, configured to actuate the gripper to transition between the unlocked state and the locked state.

In an application, the unlocker is configured to maintain the gripper in the unlocked state, the gripper being configured to automatically transition into the locked state upon removal of the unlocker from the gripper.

In an application, the unlocker includes a filament, reversibly coupled to the unlocker, and wherein tensioning of the filament transitions the gripper to the unlocked state.

In an application, the unlocker is configured to reversibly actuate the gripper to transition repeatedly between the unlocked state and the locked state.

In an application, the gripper includes a jaw that, in the locked state, clamps onto the third portion or mid-portion, and the unlocker maintains the gripper in the unlocked state by inhibiting the jaw from clamping onto the third portion or mid-portion.

In an application:
the gripper includes a jaw that, in the locked state, clamps onto the third portion or mid-portion, and
the unlocker includes a filament (e.g., a suture or wire), reversibly coupled to the jaw such that tensioning of the filament transitions the gripper into the unlocked state by pulling the jaw away from the third portion or mid-portion.

In an application:
the apparatus further includes a guide member that defines the unlocker at a distal end of the guide member,
the apparatus further includes an adjustment-facilitating tool that includes a tensioning element that is reversibly couplable to the pouch, such that while coupled to the pouch, application of a proximally-directed force to the tensioning element stretches the pouch into its stretched state, and
the adjustment-facilitating tool is advanceable along the guide member to the implant subsequent to implantation of the implant.

In an application, the apparatus further includes an adjustment-facilitating tool that includes a tensioning element that is reversibly couplable to the pouch, such that while coupled to the pouch, application of a proximally-directed force to the tensioning element stretches the pouch into its stretched state.

In an application, the apparatus further includes a guide member, reversibly coupled to the implant, and the adjustment-facilitating tool is advanceable along the guide member to the implant subsequent to implantation of the implant.

In an application, the adjustment-facilitating tool further includes a pressing element, slidably coupled to the tensioning element such that, while the tensioning element is coupled to the pouch, application of a distally-directed force to the pressing element presses the pressing element against the implant.

In an application, the pressing element maintains coupling between the tensioning element and the pouch, and withdrawal of the pressing element decouples the tensioning element from the pouch.

In an application, the pressing element is disposed coaxially around the tensioning element.

In an application, the tensioning element is disposed coaxially around the pressing element.

In an application, the pressing element is slidably coupled to the tensioning element such that, while the tensioning element is coupled to the pouch, application of the distally-directed force to the pressing element presses the pressing element against the implant body.

In an application, the pressing element is slidably coupled to the tensioning element such that, while the tensioning element is coupled to the pouch, application of the distally-directed force to the pressing element presses the pressing element against the gripper.

In an application, the pressing element is slidably coupled to the tensioning element such that, while the tensioning element is coupled to the pouch, application of the distally-directed force to the pressing element presses the pressing element against an inner surface of the pouch.

In an application, the implant further includes a coupling that is attached to the pouch, and to which the adjustment-facilitating tool is reversibly couplable.

In an application, stretching of the pouch into the stretched state contracts the implant body.

In an application, the implant body includes a fabric sleeve that defines a circumferential wall that defines a lumen, the elongate member extends along the sleeve to where the first end portion of the elongate member is coupled to the sleeve, and stretching of the pouch into the stretched state longitudinally contracts the sleeve.

In an application, the apparatus further includes a plurality of anchors, and each anchor of the plurality of anchors:
includes a tissue-coupling element and a tool-engaging head fastened to one end of the tissue-coupling element, and
is configured to be intracorporeally delivered into the lumen of the sleeve, and to anchor the sleeve to the tissue of the subject by the tissue-coupling element being driven through the circumferential wall and into the tissue.

In one embodiment, a system or apparatus can include one or more of an implant, a gripper, an elastic pouch, and an elongate member. The elastic pouch can have a first part and a second part, the first part being coupled to the gripper. The elastic pouch can be reversibly stretchable into a stretched state in which the pouch defines a stretched distance between the first part and the second part. The elastic pouch can have a contracted state toward which the pouch is elastically biased, and in which the pouch defines a contracted distance between the first part and the second part, the contracted distance being smaller than the stretched distance. In at least one state of the gripper, the elongate member is slidable through the gripper and into the pouch. The elongate member can have an end portion that is fastened to the second part of the pouch, such that, in at least the one state of the gripper, stretching of the pouch into the stretched state pulls the elongate member through the gripper and into the pouch.

In an application, the gripper includes a plurality of teeth that provide a gripping surface configured to grip the elongate member.

In an application, the first part of the pouch defines an opening into the pouch, and in the at least one state of the gripper, the elongate member in slidable through the gripper and into the pouch via the opening.

In an application, in at least one state of the gripper, the gripper inhibits sliding of the elongate member through the gripper and out of the pouch.

In an application, the at least one state of the gripper includes a unidirectional state in which the gripper facilitates sliding of the elongate member through the gripper in a first direction that is into the pouch, and inhibits sliding of the elongate member through the gripper in a second, opposite direction that is out of the pouch.

In an application, the gripper has an unlocked state in which the gripper facilitates sliding of the elongate member through the gripper in the first direction and in the second direction.

In an application, the gripper includes one or more wheels that, in the unidirectional state, grip the elongate member, each of the one or more wheels configured to rotate in only one rotational direction.

In an application, the at least one state of the gripper includes:
an unlocked state in which the gripper facilitates sliding of the elongate member through the gripper in a first direction that is into the pouch, and in a second, opposite direction that is out of the pouch, and
a locked state in which the gripper inhibits sliding of the elongate member through the gripper in the first direction and in the second direction.

In an application, the apparatus further includes an unlocker, configured to maintain the gripper in the unlocked state, the gripper configured to automatically transition into the locked state upon removal of the unlocker from the gripper.

In an application, the gripper includes a jaw that, in the locked state, clamps onto the elongate member, and the unlocker maintains the gripper in the unlocked state by inhibiting the jaw from clamping onto the elongate member.

In an application:
the apparatus further includes a guide member that defines the unlocker at a distal end of the guide member,
the apparatus further includes an adjustment-facilitating tool that includes a tensioning element that is reversibly couplable to the pouch, such that while coupled to the pouch, application of a proximally-directed force to the tensioning element stretches the pouch into its stretched state, and
the adjustment-facilitating tool is advanceable along the guide member to the implant subsequent to implantation of the implant.

In an application, the apparatus further includes an adjustment-facilitating tool that includes a tensioning element that is reversibly couplable to the pouch, such that while coupled to the pouch, application of a proximally-directed force to the tensioning element stretches the pouch into its stretched state.

In an application, the apparatus further includes a guide member, reversibly coupled to the implant, and the adjustment-facilitating tool is advanceable along the guide member to the implant subsequent to implantation of the implant.

In an application, the adjustment-facilitating tool further includes a pressing element, slidably coupled to the tensioning element such that, while the tensioning element is coupled to the pouch, application of a distally-directed force to the pressing element presses the pressing element against the implant.

In an application, the pressing element maintains coupling between the tensioning element and the pouch, and withdrawal of the pressing element decouples the tensioning element from the pouch.

In an application, the pressing element is disposed coaxially around the tensioning element.

In an application, the tensioning element is disposed coaxially around the pressing element.

In an application, the pressing element is slidably coupled to the tensioning element such that, while the tensioning element is coupled to the pouch, application of the distally-directed force to the pressing element presses the pressing element against the implant body.

In an application, the pressing element is slidably coupled to the tensioning element such that, while the tensioning element is coupled to the pouch, application of the distally-directed force to the pressing element presses the pressing element against the gripper.

In an application, the pressing element is slidably coupled to the tensioning element such that, while the tensioning element is coupled to the pouch, application of the distally-directed force to the pressing element presses the pressing element against an inner surface of the pouch.

In an application, the implant further includes a coupling that is attached to the pouch, and to which the adjustment-facilitating tool is reversibly couplable.

Various methods of using the systems and apparatuses described herein are possible. For example, a method for use with an implant having an implant body and a pouch, can include one or more of the following steps:
implanting the implant in a heart of a subject; and
contracting the implant body by stretching the pouch into a stretched state by pulling on part of the pouch using a tensioning element.

In an application, implanting the implant includes transluminally delivering the implant and anchoring the implant to a valve annulus of the heart, and contracting the implant body includes transluminally contracting the implant.

In an application, implanting the implant includes transluminally delivering a plurality of anchors to the implant, and sequentially driving the anchors through the implant body and into tissue of the heart.

In an application, the implant body can include an annuloplasty ring structure, other annuloplasty device structure, or other cinchable/tensionable structure, and implanting the implant can include anchoring the implant body (e.g., the annuloplasty ring structure, or other structure) to an annulus.

In an application, contracting the implant body includes stretching the pouch into the stretched state by pulling on the part of the pouch using the tensioning element, while simultaneously providing an opposing force via a pressing element that abuts the implant.

In an application, the method further includes advancing the tensioning element to the implant subsequently to implanting the implant, and prior to contracting the implant.

In an application, advancing the tensioning element to the implant includes advancing the tensioning element over a guide member that is reversibly coupled to the implant.

In an application, the method further includes decoupling the guide member from the implant subsequently to advancing the tensioning element to the implant.

In an application, the method further includes, subsequently to stretching the pouch into the stretched state, allowing the pouch to elastically contract toward a contracted state without reversing the contraction of the implant body.

In an application, the implant includes an elongate member and a gripper, the elongate member extending from the part of the pouch, though the gripper, and into the implant body, and contracting the implant body includes drawing the elongate member through the gripper and into the pouch by stretching the pouch into the stretched state.

In an application, the method further includes, subsequently to stretching the pouch into the stretched state, allowing the pouch to elastically contract toward a contracted state without allowing the elongate member to move back out of the pouch and through the gripper.

In an application, drawing the elongate member through the gripper and into the pouch includes drawing the elongate member through the gripper and into the pouch while maintaining the gripper in an unlocked state.

In an application, the method further includes, subsequently to drawing the elongate member through the gripper and into the pouch, transitioning the gripper into a locked state.

In an application, transitioning the gripper into the locked state includes removing an unlocker from the gripper such that the gripper automatically transitions into the locked state.

Additional components/features and additional steps described elsewhere herein can also be used in the examples above.

The present invention will be more fully understood from the following detailed description of applications thereof, taken together with the drawings, in which:

### BRIEF DESCRIPTION OF THE DRAWINGS

FIGS. 1A-C are schematic illustrations of an adjustment system, in accordance with some applications of the invention;

FIGS. 2A-D and 3A-D are schematic illustrations of respective embodiments of an adjustment-facilitating tool being used with the adjustment system to adjust an implant, in accordance with some applications of the invention;

FIGS. 4A-D are schematic illustrations of the use of a guide member to advance the adjustment-facilitating tool to the implant, in accordance with some applications of the invention; and

FIGS. 5, 6A-B, and 7A-B are schematic illustrations of respective embodiments of a gripper, in accordance with some applications of the invention.

### DETAILED DESCRIPTION OF EMBODIMENTS

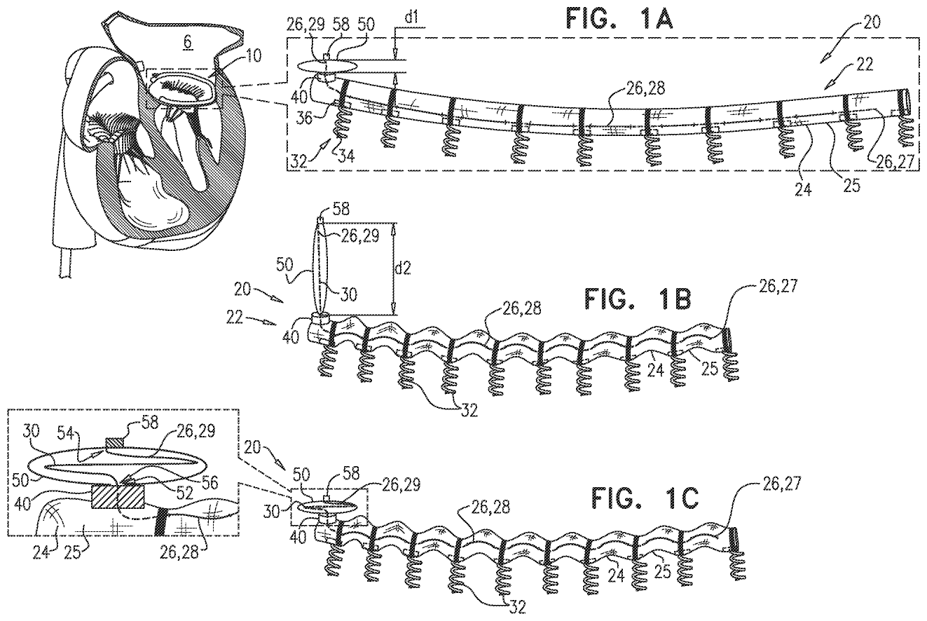

Reference is made to FIGS. 1A-C, which are schematic illustrations of an adjustment system 20, in accordance with some applications of the invention.

System 20 can comprise an implant 22 that comprises an implant body 24, and the system is for adjustment of the implant body. The implant 22 and/or implant body 24 shown can take a variety of forms and those depicted in the various figures herein are merely exemplary. For some applications, and as generally described herein, implant body 24 can be an annuloplasty ring structure, annuloplasty/annulus adjustment structure, other annuloplasty device structure, or other cinchable/tensionable structure. Implant body 24 can comprise a flexible sleeve 25 that has a wall or circumferential wall (e.g., which can be made of a fabric, such as a polyethylene terephthalate fabric, e.g., Dacron™) that circumscribes a longitudinal axis of the sleeve, thereby defining a longitudinal lumen. System 20 (e.g., implant 22 thereof) can further comprise an elongate member 26. Elongate member 26 can comprise one or more strands of metal or polymer, optionally coated with a low-friction coating, such as polytetrafluoroethylene (PTFE). Implant body 24 can be configured to be placed partially or completely around a cardiac valve annulus 10 (e.g., a mitral valve annulus, tricuspid valve annulus, etc.). Implant body 24 can be secured or anchored in place at the valve annulus in a variety of ways, including by suturing the implant body 24 to the valve annulus, e.g., by parachuting similar to a surgical annuloplasty ring or otherwise. Implant body 24 can be configured to be anchored in place using a plurality of (e.g., 5-20) tissue anchors 32. The tissue anchors 32 can have a variety of features, for example, each tissue anchor 32 can comprise a tissue-coupling element 34, and a tool-engaging head 36 fastened to one end of the tissue-coupling element. Following introduction of implant body 24 into the subject, each anchor 32 can be sequentially intracorporeally delivered into the lumen of the sleeve, and its tissue-coupling element 34 can be driven from the interior lumen or from one side through the circumferential wall and into tissue of the valve annulus, thereby anchoring the implant body 24 or sleeve to the valve annulus. After the implant 22 and/or implant body 24 are secured or anchored to the valve annulus, longitudinal contraction of implant body 24, facilitated by system 20, can be used to circumferentially tighten the valve annulus, thereby improving coaptation of the valve leaflets, and reducing regurgitation.

For some applications, the implant structure of implant body 24 can be, or share features with, mutatis mutandis, any of the implant structures described in one or more of the following publications, which are incorporated herein by reference. The relevant features of the system herein may be substituted for adjustment features in these references. For some applications, implant 22 is implanted as described in one or more of these publications, mutatis mutandis:
PCT application publication WO 2010/128503 to Zipory et al.
PCT application publication WO 2012/176195 to Gross et al.
PCT application publication WO 2013/069019 to Sheps et al.
PCT application publication WO 2014/064694 to Sheps et al.
U.S. Pat. No. 8,926,696 to Miller et al.
U.S. Pat. No. 8,353,956 to Miller et al.

FIGS. 1A-C illustrate a generalization of system 20. More specific embodiments are described with reference to later figures.

Implantable apparatus or system 20 can comprise a gripper 40 and a pouch 50 (e.g., an elastic pouch) or other container. Pouch 50 can have a first part 54 and a second part 56, and has a contracted state (see FIGS. 1A and 1C) toward which the pouch is biased (e.g., elastically biased, shape-memory biased, etc.). The pouch 50 can have one or multiple contracted states. In the contracted state, the pouch can be configured to define a contracted distance d1 between the first part and the second part. Pouch 50 can be reversibly stretchable into a stretched state (FIG. 1B) in which the pouch defines a stretched distance d2 between first part 54 and second part 56. Distance d1 is smaller than distance d2. For example, distance d1 may be 1-12 mm (e.g., 1-7 mm, such as 2-6 mm), whereas distance d2 may be 20-100 mm (e.g., 30-80 mm, such as 40-70 mm).

Implantable apparatus or system 20 can also include an elongate member 26 that can extend into the pouch 50. Elongate member can be arranged such that it extends through a gripper 40, and then into pouch 50 via an opening 52. Opening 52 can be defined by second part 56 of the pouch. A first end portion 27 of elongate member 26 can be coupled to implant body 24. A second end portion 29 of elongate member 26 can be fastened to first part 54 of the pouch. Second part 56 can be defined by the location of opening 52, and first part 54 can be defined by the location of the fastening of elongate member 26 to pouch 50.

A third portion or mid-portion 28 of elongate member 26 can be between end portions 27 and 29. Mid-portion or third portion 28 can extend through gripper 40. Gripper 40 is configured (i) to facilitate tensioning of the elongate member, and (ii) to subsequently maintain the tension on the elongate member. As described in more detail hereinbelow, gripper 40 can have at least one state in which third portion or mid-portion 28 is slidable through the gripper and into pouch 50 via opening 52. For example, gripper 40 may facilitate one-way sliding of portion 28 therethrough. Alternatively or additionally, gripper 40 can have (i) an unlocked state in which it facilitates sliding of portion 28 therethrough, and (ii) a locked state in which it inhibits the sliding of the third portion or mid-portion.

Implantable apparatus or system 20 can also comprise an implant 22. FIG. 1A shows implant 22 subsequent to its implantation, and before its adjustment (i.e., contraction). Pouch 50 is shown in a contracted state (e.g., relaxed state). FIG. 1B shows pouch 50 having been reversibly stretched into a stretched state. Because second end portion 29 is fastened to first part 54, this stretching pulls on elongate member 26, drawing it (e.g., third portion or mid-portion 28 thereof) through gripper 40 and opening 52, into pouch 50, thereby longitudinally contracting implant body 24, e.g., by reducing the length of the elongate member disposed within the implant body. For applications in which implant body 24 is an annuloplasty ring structure or other annuloplasty device structure implanted around the valve annulus (e.g., all or a portion thereof), this longitudinal contraction circumferentially tightens the valve annulus, thereby improving coaptation of the valve leaflets, and reducing or eliminating regurgitation. It is to be understood that, during this step, gripper 40 can be in at least one state in which portion 28 is slidable through the gripper and into pouch 50 via opening 52. Optionally, pouch 50 and gripper 40 can be integral with or separate from implant 22. The implants or implant bodies described herein can have an open or closed (e.g., a closed ring) configuration and can be configured for transvascular or transcatheter implantation and/or surgical implantation.

FIG. 1B does not show how pouch 50 is stretched. Some examples of techniques for stretching pouch 50 are described in further detail hereinbelow. However, FIG. 1B does show a coupling 58 via which a tool is couplable to pouch 50 for stretching (e.g., pulling on) the pouch. For some applications, and as shown throughout this patent application, second end portion 29 of elongate member 26 can be fastened to first part 54 of pouch 50 by being fastened to coupling 58. The position of coupling 58 may thereby define or be first part 54 of pouch 50.

Subsequently, pouch 50 is allowed to return toward its contracted state (FIG. 1C), but gripper 40 can be designed or configured to inhibit or prevent elongate member 26 from sliding back through gripper 40 and into implant body 24. For example, and as described hereinbelow, gripper 40 may only facilitate one-way movement of member 26 therethrough, or may have been locked subsequently to the stretching of pouch 50. Because elongate member 26 is inhibited or prevented from sliding back through gripper 40, implant body 24 remains in its reduced length, and the valve annulus remains at its repaired size.

For some applications, pouch 50 can be made from or comprise an elastomer. For some applications, pouch 50 can comprise one or more strands of elastic and/or shape memory material such as Nitinol, e.g., optionally, threaded into the pouch so as to provide the pouch with its elastic nature.

Pouch 50 is typically distinct from an inflatable balloon. For some applications, pouch 50 can be partially permeable, allowing blood (or components thereof) to enter the pouch over time, and facilitating clotting and/or tissue growth therein. It is believed that this may facilitate maintenance of the tension applied to elongate member 26, and thereby maintenance of the contraction of implant body 24.

For some applications, pouch 50 can be impermeable to blood and/or components thereof. It is believed that this may facilitate readjustment of implant 22 subsequent to its implantation and initial contraction.

While a pouch might not be used, the pouch is beneficial to contain and restrict movement of any excess portion of the elongate member. For example, in the absence of pouch 50 (e.g., adjustment by pulling directly on end portion 29), the resulting excess 30 of elongate member 26 outside of implant body 24 may disadvantageously move freely within the body (e.g., within left atrium 6), with potentially deleterious effects. The elastic return of pouch 50 to its contracted state can compress excess 30 of elongate member 26, such that it is tidily confined. It is believed that this can advantageously avoid excess 30 being free within the heart, while obviating a potential need to remove (e.g., by cutting) excess 30.

For some applications, pouch 50, gripper 40, and implant body 24 can be coupled together such that, from proximal end portion 29, until at least implant body 24, elongate member 26 is not exposed to the body of the subject. That is, the pouch and gripper isolate the elongate member from the body of the subject. For some applications, more distal portions of elongate member 26 are exposed to the body of the subject, e.g., if elongate member 26 weaves in and out of implant body 24 (e.g., the circumferential wall of sleeve 25).

Although system 20 is generally described herein as including implant 22, it is to be noted that the scope of the invention does not require the system 20 to include the implant or any other component. For example, system 20 may be provided alone or unattached or uncombined with an implant, such that it can be later attached to and/or used with a different implant, e.g., a third-party implant. In one embodiment, system 20 can comprise pouch 50, gripper 40, elongate member 26, and one or more anchors attached to the elongate member, e.g., without a separate implant body.

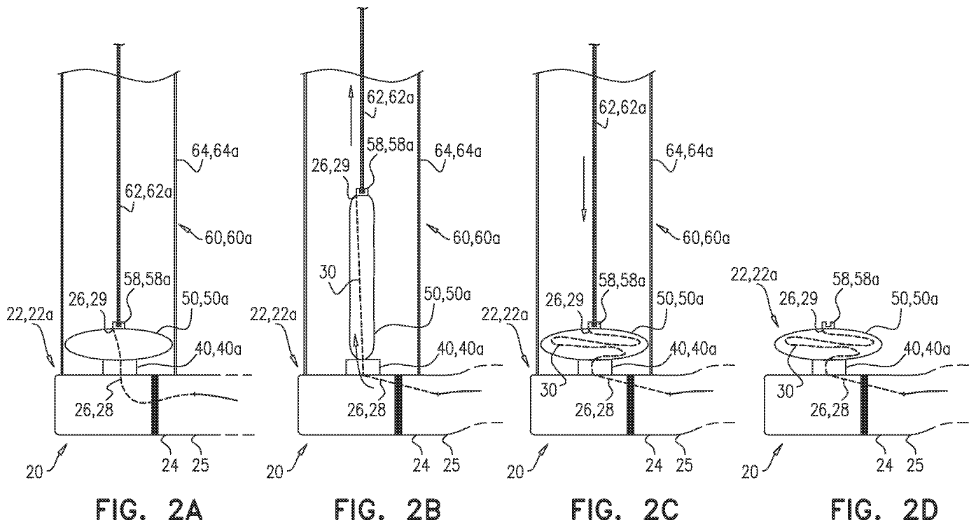

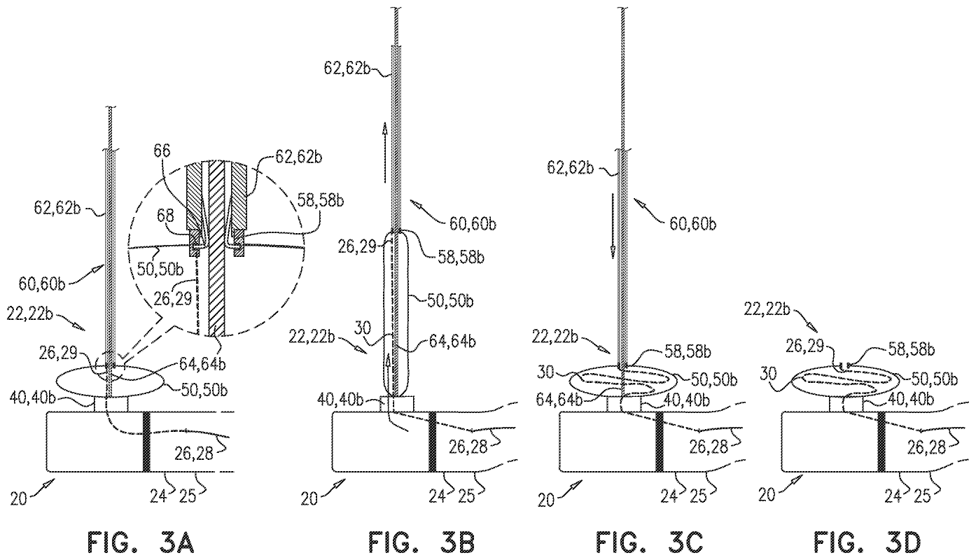

Reference is made to FIGS. 2A-D and 3A-D, which are schematic illustrations of exemplary embodiments of adjustment-facilitating tool 60 being used with system 20 to adjust (e.g., contract) implant 22, in accordance with some applications of the invention. Various embodiments of tool 60 can comprise a tensioning element 62, and/or can comprise a pressing element 64.

The use of common reference numerals is intended to indicate commonality between elements of different embodiments, e.g., whereby an element of one embodiment serves the same primary function as the corresponding element of another embodiment. Suffixes (e.g., a, b, etc.) are used to uniquely identify the elements of a particular embodiment. For example, the embodiments of FIGS. 2A-D and 3A-D are depicted as each including a tool 60 (tools 60a and 60b, respectively) that comprises a tensioning element 62 (elements 62a and 62b). Despite the described differences between tools 60a and 60b, and between elements 62a and 62b, both tools are used to contract their respective implant 22, and both tensioning elements are coupled to their respective pouch 50, and are used to apply a proximally-directed force to (i.e., to pull on) the pouch to stretch it.

FIGS. 2A-D show an exemplary embodiment in which a tool 60a (an embodiment of tool 60) comprises a tensioning element 62a (an embodiment of tensioning element 62) and a pressing element 64a (an embodiment of pressing element 64), and an implant 22a (which can be the same as or similar to implant 22 or can be any implant that requires cinching or tightening from a remote or removed location) comprises a gripper 40a (an embodiment of gripper 40), an elastic pouch 50a (an embodiment of pouch 50), and a coupling 58a (an embodiment of coupling 58).

For some applications, implant 22 (e.g., implant 22a) can be delivered and implanted while at least part of tool 60 (e.g., tool 60a) is coupled to the implant. For example, implant 22a can be delivered and implanted while (i) tensioning element 62a is coupled to coupling 58a, and (ii) pressing element 64a abuts implant body 24. Alternatively, implant 22a can be delivered and implanted while (i) tensioning element 62a is coupled to coupling 58a, and (ii) pressing element 64a is proximal to the implant (e.g., within a delivery catheter or outside of the subject), and is subsequently advanced over and along the tensioning element to the implant. Optionally, another arrangement could also be used.

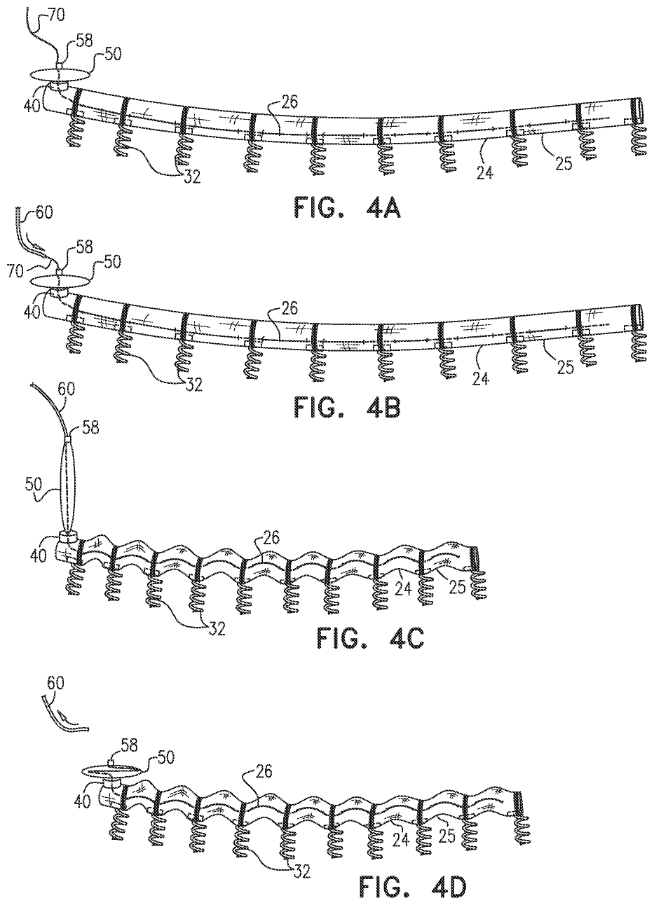

For some applications, implant 22 (e.g., implant 22a) is delivered and implanted without tool 60 (e.g., tool 60b) coupled to the implant, and one or more components of the tool are subsequently advanced to the implant (e.g., over and along a dedicated guide wire or guide member) and coupled to the implant, e.g., as described with reference to FIGS. 4A-D, mutatis mutandis.

While tensioning element 62a is coupled to coupling 58a, the tensioning element can be pulled proximally to reversibly stretch pouch 50a. Stretching the pouch can draw elongate member 26 (e.g., portion 28 thereof) through gripper 40a and into the pouch (FIG. 2B). This can be performed while pressing element 64 provides an opposing force against implant 22 (e.g., against implant body 24) in order that the pulling not pull the implant away from the tissue at which it is implanted. Pressing element 64a can be tubular and/or configured in a variety of shapes. Pressing element 64a can be disposed around tensioning element 62a (e.g., coaxially). Pressing element 64a or at least at its distal end can be wider than pouch 50a, such that it can press against the implant without interfering with the stretching of the pouch. Optionally, the pressing element could be configured to pass to one or more sides of the pouch to contact the implant, e.g., without surrounding the pouch.

After the implant and/or implant body 24 has been sufficiently contracted—which can be determined by imaging of the implant (e.g., using fluoroscopy, etc.), the anatomy, and/or blood flow (e.g., using echocardiography, ultrasound, etc.)—pouch 50a can be allowed to return to its contracted state (FIG. 2C). Gripper 40a can be configured to inhibit elongate member 26 from sliding back through the gripper and into implant body 24. For example, the gripper 40a and/or other grippers described herein can have friction resistance, a latch, valve, clamp, etc. that can allow movement of the member in one direction, but not the other direction. Tool 60a can be decoupled from implant 22a (e.g., element 62a can be decoupled from coupling 58a), and withdrawn from the subject (FIG. 2D). For some applications, and as shown, pouch 50a is allowed to return to its contracted state by moving element 62a distally, and/or by releasing tension on element 62a such that the elastically-contracting pouch pulls element 62a distally. For some applications, pouch 50a is allowed to return to its contracted state by decoupling tool 60a, including moving element 62a, from implant 22a, e.g., skipping the step shown in FIG. 2C.

FIGS. 3A-D show an embodiment in which a tool 60b (an embodiment of tool 60) comprises a tensioning element 62b (an embodiment of tensioning element 62) and a pressing element 64b (an embodiment of pressing element 64), and an implant 22b (e.g., the same as or similar to implant 22, 22a or any implant that needs to be contracted or tensioned) comprises a gripper 40b (an embodiment of gripper 40), an elastic pouch 50b (an embodiment of pouch 50), and a coupling 58b (an embodiment of coupling 58).

For some applications, implant 22b is delivered and implanted while at least part of tool 60b is coupled to the implant. For example, implant 22b can be delivered and implanted while tensioning element 62b is coupled to coupling 58b. Optionally, implant 22a can be delivered and implanted while (i) pressing element 64b is coupled to the implant (e.g., to gripper 40b thereof), and (ii) tensioning element 62b is proximal to the implant (e.g., within a delivery catheter or outside of the subject), and is subsequently advanced over and along the pressing element to the implant.

For some applications, implant 22b is delivered and implanted without tool 60b coupled to the implant, and one or more components of the tool are subsequently advanced to the implant (e.g., over and along a dedicated guide wire or guide member) and coupled to the implant, e.g., as described with reference to FIGS. 4A-D, mutatis mutandis.

While tensioning element 62b is coupled to coupling 58b, the tensioning element can be pulled proximally to reversibly stretch pouch 50b. Stretching pouch 50b can draw elongate member 26 (e.g., portion 28 thereof) through gripper 40b and into the pouch (FIG. 3B). This can be performed while pressing element 64b provides an opposing force against implant 22b in order that the pulling not pull the implant away from the tissue at which it is implanted. In contrast to the arrangement of elements 62a and 64a as shown in FIGS. 2A-2D, tensioning element 62b is depicted as being tubular and disposed around pressing element 64b (e.g., coaxially). Pressing element 64b can be configured and arranged to extend distally from tensioning element 62b, and through pouch 50b, e.g., such that is abuts and presses against the inner surface of the pouch and/or against gripper 40b, and such that it can provide the opposing force without interfering with the stretching of the pouch. Coupling 58b can be shaped to define an opening through which pressing element 64b extends or can extend into pouch 50b. When pouch 50b is stretched, coupling 58b can slide proximally along pressing element 64b.

After the implant 22 and/or implant body 24 has been sufficiently contracted, pouch 50b is allowed to return to its contracted state (FIG. 3C). Gripper 40b can be the same as or similar to other grippers described herein and can be configured to inhibit elongate member 26 from sliding back through the gripper and into implant body 24. Tool 60b can be decoupled from implant 22b (e.g., element 62b can be decoupled from coupling 58b), and withdrawn from the subject (FIG. 3D). For some applications, and as shown, pouch 50b can be allowed to return to its contracted state by moving element 62b distally, and/or by releasing tension on element 62b such that the elastically-contracting pouch pulls element 62b distally. For some applications, pouch 50b is allowed to return to its contracted state by decoupling tool 60b, including element 62b, from implant 22b, e.g., skipping the step shown in FIG. 3C.

For some applications, pressing element 64 maintains the coupling between tensioning element 62 and coupling 58. For example, with reference to FIGS. 3A-D, the presence of pressing element 64b within coupling 58b may hold a detent 66 defined by tensioning element 62b within a recess 68 defined by coupling 58b (or a detent defined by the coupling within a recess defined by the tensioning element). For example, the pressing element and/or detent can be configured or designed such that, upon removal of the pressing element, the detent automatically retracts from the recess, decoupling the tensioning element from the coupling.

Reference is again made to FIGS. 2A-D and 3A-D. Optionally, tool 60 can comprise an extracorporeal controller (not shown), which can comprise at least one handle, at the proximal end of elements 62 and 64. For some applications, the length of elongate member 26 (e.g., of portion or mid-portion 28) that slides proximally through gripper 40 and into pouch 50 is proportional (e.g., equal) to the amount by which implant body 24 contracts. Therefore, for some applications, the length by which implant body 24 contracts can be proportional (e.g., equal) to the distance moved by tensioning element 62 relative to pressing element 64. For some such applications, the amount of contraction of implant body 24 can be indicated by control members of the extracorporeal controller. For example, actuation of a control member (e.g., turning of one or more knobs) may cause movement between elements 62 and 64, and the amount that the control member has been actuated (e.g., the amount that the one or more knobs have been turned) can be indicated by a dial. For example, thereby indicating the amount of relative movement between elements 62 and 64, and thereby the amount of contraction of implant body 24.

Reference is now made to FIGS. 4A-D, which are schematic illustrations of the use of a guide member 70 (e.g., which can be a guide wire or other guide) to advance adjustment-facilitating tool 60 to implant 22 and/or to pouch 50, in accordance with some applications of the invention. For some applications, the implant can be implanted without an adjustment tool (e.g., tool 60) coupled thereto, and the tool can be subsequently advanced to the implant in order to adjust the implant. For such applications, implant 22 can be implanted with a guide member 70 coupled thereto (FIG. 4A). Subsequent to implantation, tool 60 can be advanced along (optionally over) guide member 70 (FIG. 4B), then tensioning element 62 can be coupled to pouch 50 (e.g., via coupling 58). Tool 60 can then be used to adjust implant 22 (e.g., to longitudinally contract the implant), e.g., by reversibly stretching pouch 50 (FIG. 4C). Pouch 50 can then be allowed to return to its contracted state, and tool 60 can be decoupled from implant 22 (FIG. 4D). As shown, guide member 70 can be decoupled from implant 22 subsequent to adjustment of the implant. This can be achieved by any suitable technique, such as unscrewing, unlocking a lock, etc., and can be performed using tool 60.

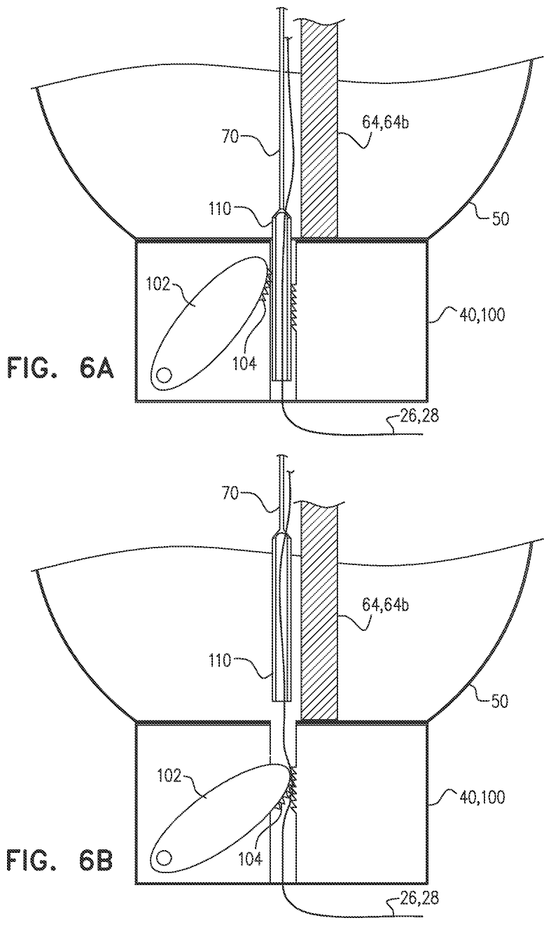

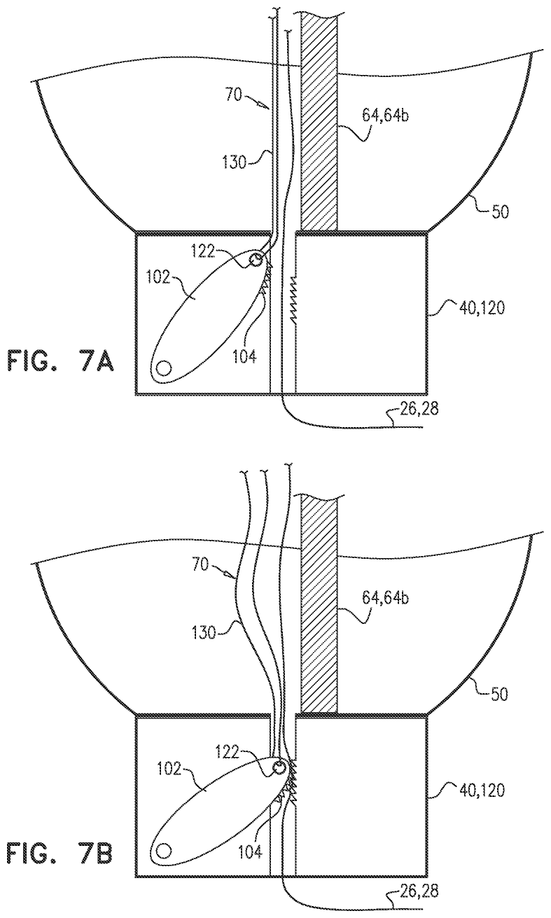

For some applications, guide member 70 is coupled to implant 22 at coupling 58. For some applications, guide member 70 is coupled to implant 22 at gripper 40 (e.g., as described with reference to FIGS. 6A-B). For some such applications, guide member 70 extends through pouch 50 to gripper 40. Other coupling options are also possible.

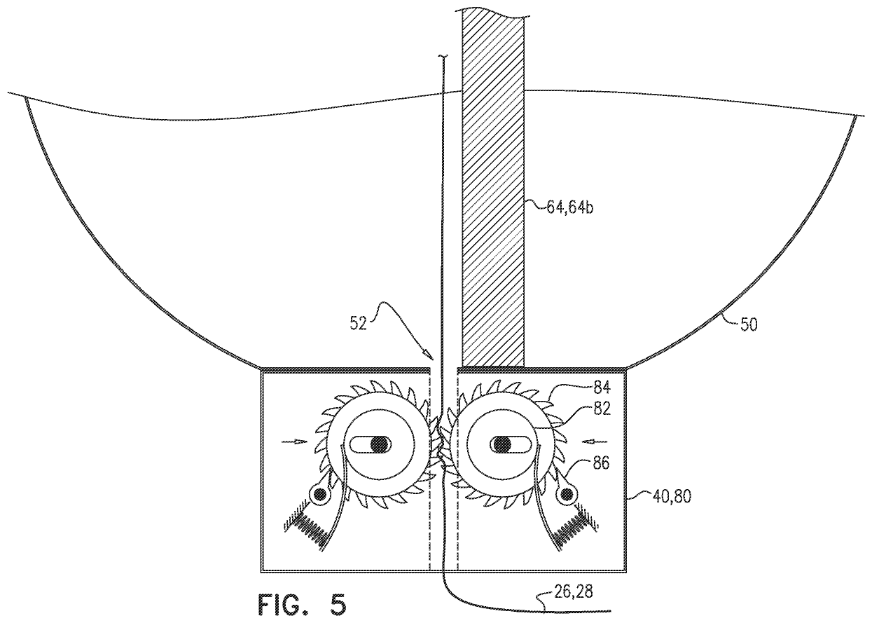

Reference is now made to FIGS. 5, 6A-B, and 7A-B which are schematic illustrations of exemplary embodiments of gripper 40. As described hereinabove, gripper 40 can allow for unidirectional movement/sliding or have at least one state in which third portion or mid-portion 28 of elongate member 26 is slidable through the gripper and into pouch 50 via opening 52. The gripper 40 can be configured such that the portion 28 and/or elongate member 26 is not slidable from the pouch back in to the implant. For example, gripper 40 may facilitate only one-way sliding of portion 28 therethrough. Optionally, it is also possible to configure gripper 40 to allow movement or sliding of the portion 28 and/or elongate member 26 into and out of the gripper 40 in both directions until the gripper is locked, clamped, or otherwise secured to prevent movement or sliding of the portion 28 and/or elongate member 26 therethrough. For example, gripper 40 may have (i) an unlocked state in which it facilitates sliding of portion 28 therethrough, and (ii) a locked state in which it inhibits the sliding of the portion/mid-portion.

FIG. 5 shows an exemplary embodiment 80 of gripper 40, which has a unidirectional state in which the gripper facilitates one-way sliding of portion 28 therethrough. Gripper 80 can comprise one or more wheels 82, each wheel comprising a gripping surface 84 (e.g., comprising a plurality of teeth or comprising other frictional element(s)) that can grip portion 28. Wheels 82 can be configured to rotate in only one rotational direction, and thereby facilitate movement of portion 28 in a first direction through the gripper into pouch 50, but not in a second direction through the gripper out of the pouch. For example, gripper 80 may comprise a pawl 86 that allows rotation of wheel 82 in only one rotational direction. Optionally, the one or more wheels could be configured to allow rotation in both directions until locked or secured to prevent further movement.

FIGS. 6A and 6B show an exemplary embodiment 100 of gripper 40 in which the gripper has (i) an unlocked state in which it facilitates sliding of portion 28 in either direction therethrough, and (ii) a locked state in which it inhibits the sliding of the portion in either direction. Optionally, the gripper can be biased to assume the locked state.

For some applications, gripper 100 can comprise at least one locking element 102 (e.g., a jaw, latch, contact edge, etc.) that, in the locked state, clamps onto portion 28 or elongate member 26. The jaw 102 (or other locking element) can have a gripping surface 104, such as one or more teeth or other frictional feature(s), that frictionally engages or grips portion 28 when the locking element or jaw contacts or clamps onto the portion of elongate member 26.

The system can also comprise an unlocker 110, which can be configured to maintain the gripper in an unlocked state. Optionally, gripper 100 can be configured to automatically transition into a locked state upon removal of unlocker 110 from the gripper. As shown, for example, in FIGS. 6A and 6B, unlocker 110 can be shaped to define a partial tube that inhibits (e.g., obstructs) jaw 102 (or other locking element) from clamping on to portion 28 and allows the portion to slide through the cavity defined by the partial tube. Because the unlocker defines only a partial tube, it is not threaded onto elongate member 26, and can therefore be removed from the implant after the gripper is locked. Unlocker 110 thereby serves as an actuator of gripper 100 (e.g., of jaw 102 thereof), via which the gripper is transitionable between its unlocked and locked states.

For some applications, and as shown, unlocker 110 can be defined by, or can be coupled to, the distal end of guide member 70. For such applications, guide member 70 can continue distally past coupling 58 and into pouch 50. Optionally, unlocker 110 can be distinct from guide member 70, and/or can be used in systems that do not include guide member 70.

FIGS. 7A and 7B show another exemplary embodiment 120 of gripper 40 in which the gripper has (i) an unlocked state in which it facilitates sliding of portion 28 in either direction therethrough, and (ii) a locked state in which it inhibits the sliding of the portion in either direction. Gripper 120 is typically identical to gripper 100 except where noted.

Gripper 120 is configured to be transitioned between its locked and unlocked states via an unlocker 130, which thereby serves as an actuator of gripper 120 (e.g., of jaw 102 thereof). In contrast to unlocker 110, unlocker 130 actuates gripper 120 by pulling on jaw 102, thereby pulling the jaw away from mid-portion 28 of elongate member 26. Unlocker 130 is reversibly coupled to jaw 102, such that when the unlocker is pulled proximally (e.g., tensioned), the jaw is opened, thereby transitioning the gripper into its unlocked state. The gripper can be biased to assume the locked state (e.g., in the absence of pulling by unlocker 130).

For some applications, and as shown, unlocker 130 comprises a filament (e.g., a wire or suture), and is reversibly coupled to jaw 102 by being looped around part of the jaw. For example, and as shown, the filament may be coupled to the jaw by being looped through an eyelet 122 that is coupled to or defined by the jaw. For some such applications, both ends of the filament are disposed outside of the heart (e.g., outside of the subject), and the unlocker is tensioned by pulling on both ends. For some such applications, decoupling of the unlocker from the gripper is achieved by releasing one end of the filament and pulling on the other end of the filament, causing the released end to travel toward the jaw and through the eyelet, thereby unthreading the filament from the eyelet.

For some applications, and as shown, unlocker 130 (e.g., proximal portions thereof) also serve as guide member 70, e.g., as described hereinabove, mutatis mutandis. This may alternatively be viewed as the unlocker being defined at a distal end of the guide member.

The use of unlocker 130 allows gripper 120 to be reversibly locked (e.g., locked, unlocked, and relocked), thereby further facilitating controlled contraction of the implant.

Other grippers described or shown herein can also include an unlocker, for example, gripper 80 can also have an unlocked state, and can be provided with an unlocker that inhibits wheels 82 from engaging portion 28 in one position and can be moved to another position to allow wheels 82 to engage portion 28.

The gripper 40 is not limited to what has been particularly shown and described hereinabove. For example, although gripping surfaces 84 and 104 are both shown as comprising teeth, other gripping surfaces (e.g., high friction surfaces) may be used. For some applications, the gripper can comprise a radially-contracting element (e.g., a helical spring) that is configured to grip the elongate member by being biased to radially contract, e.g., upon removal of an unlocker.

Grippers 40, 80, 100, and 120 etc. can be used in combination with any of the implants described herein, mutatis mutandis. For example, gripper 40a of implant 22a, or gripper 40b of implant 22b, can comprise gripper 80, 100, 120, or another gripper, mutatis mutandis. The grippers may alternatively or additionally be used to facilitate tensioning of, and/or maintenance of tension on, elongate members of other implants, including implants that do not comprise pouch 50. Such implants include adjustable annuloplasty bands, adjustable annuloplasty rings, and adjustable prosthetic chordae tendineae.

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Methods can include steps described above in various orders and combinations. Features and characteristics of one embodiment can be combined or incorporated into other embodiments.

1. Apparatus for use at a valve of a heart of a subject, the apparatus comprising:
an annuloplasty implant, configured for transluminal delivery to the valve, and comprising:
an elongate member having a proximal end, and a distal portion that includes a distal end of the elongate member; and
a plurality of anchors, distributed along the elongate member, the plurality of anchors including a distal-most anchor and a proximal-most anchor;
a gripper that comprises a locking element that, in a locked state thereof, inhibits sliding of the elongate member through the gripper by clamping the elongate member;
an unlocker; and
a tool, configured to:
while (i) the elongate member extends proximally from the proximal-most anchor and through the gripper, and (ii) the unlocker is positioned within the gripper in a manner that obstructs the locking element from assuming the locked state:
advance transluminally toward the proximal-most anchor, and
tension at least the distal portion of the elongate member by pulling the elongate member proximally through the gripper while the unlocker obstructs the locking element from assuming the locked state,
wherein the locking element is configured to clamp the elongate member responsively to withdrawal of the unlocker from the gripper.

2. The apparatus according to claim 1, wherein the unlocker is shaped to define a partial tube.

3. The apparatus according to claim 1, further comprising a container, coupled to the gripper, wherein the tool is configured to, after tensioning the elongate member, release the elongate member such that a proximal portion of the elongate member, disposed proximally from the gripper, is confined by the container.

4. The apparatus according to claim 3, wherein the container is configured to allow blood to enter the container, and to reach the proximal portion of the elongate member confined by the container.

5. The apparatus according to claim 3, wherein the container is a pouch.

6. The apparatus according to claim 3, wherein:
the container has a first state and a second state, and
the tool is configured to:
maintain the container in the first state, and
after releasing the elongate member, trigger the container to transition into the second state, the proximal portion of the elongate member confined by the container while the container is in the second state.

7. The apparatus according to claim 6, wherein the tool is configured to reversibly engage the container.

8. The apparatus according to claim 6, wherein the container is elastic, the first state is a stretched state, and the second state is a contracted state.

9. The apparatus according to claim 6, wherein the container is elastically biased to assume the second state.

10. The apparatus according to claim 6, wherein the container comprises a shape memory material that configures the container to assume the second state.

11. A method for use at a valve of a heart of a subject, the method comprising:
transluminally delivering to the valve:
an elongate member having a proximal end, and a distal portion that includes a distal end of the elongate member; and
a plurality of anchors, distributed along the elongate member, the plurality of anchors including a distal-most anchor and a proximal-most anchor;
while (i) the elongate member extends proximally from the proximal-most anchor and through a gripper, and (ii) an unlocker is positioned within the gripper in a manner that obstructs a locking element of the gripper from clamping onto the elongate member:
advancing a tool transluminally toward the proximal-most anchor; and
subsequently, using the tool to facilitate tensioning of at least the distal portion of the elongate member by pulling the elongate member proximally through the gripper while the unlocker obstructs the locking element from clamping onto the elongate member; and
subsequently, clamping, via the locking element, the elongate member responsive to withdrawal of the unlocker from the gripper.

12. The method according to claim 11, wherein:
the unlocker is shaped to define a partial tube, and
using the tool to facilitate tensioning of at least the distal portion of the elongate member comprises using the tool to pull the elongate member proximally through the gripper while the partial tube is disposed within the gripper and obstructs the locking element from clamping onto the elongate member.

13. The method according to claim 11, wherein the method further includes, subsequently to using the tool to facilitate tensioning of at least the distal portion of the elongate member, releasing the elongate member such that a proximal portion of the elongate member, disposed proximally from the gripper, is confined within a container that is coupled to the gripper.

14. The method according to claim 13, wherein:
the container is configured to allow blood to enter the container, and to reach the proximal portion of the elongate member confined within the container, and
releasing the elongate member such that the proximal portion of the elongate member is confined within the container, comprises releasing the elongate member such that the proximal portion of the elongate member is confined within the container that is configured to allow blood to enter the container and reach the proximal portion of the elongate member disposed therein.

15. The method according to claim 13, wherein the container is a pouch, and wherein releasing the elongate member such that the proximal portion of the elongate member is confined by the container, comprises releasing the elongate member such that the proximal portion of the elongate member is confined by the pouch.

16. The method according to claim 13, wherein:
the container has a first state and a second state,
using the tool to facilitate tensioning of at least the distal portion of the elongate member comprises using the tool to facilitate tensioning of at least the distal portion of the elongate member while the tool maintains the container in the first state, and
releasing the elongate member comprises releasing the elongate member such that the container transitions into the second state, the proximal portion of the elongate member being confined by the container while the container is in the second state.

17. The method according to claim 16, wherein:
the container is elastic;
the first state is a stretched state, and tensioning at least the distal portion of the elongate member while the tool maintains the container in the first state comprises tensioning at least the distal portion of the elongate member while the tool maintains the container in the stretched state; and
the second state is a contracted state, and releasing the elongate member such that the container transitions into the second state comprises releasing the elongate member such that the container transitions into the contracted state.

18. The method according to claim 16, wherein:
the container is elastically biased to assume the second state, and
releasing the elongate member comprises releasing the elongate member such that the elastic bias of the container transitions the container into the second state.

19. The method according to claim 16, wherein:
the container comprises a shape memory material that configures the container to assume the second state, and
releasing the elongate member comprises releasing the elongate member such that the shape memory material transitions the container into the second state.
